(19) United States
(12) Patent Application Publication
(10) Pub. No.: US 2020/0160992 A1
(43) Pub. Date: May 21, 2020

(54) PSYCHOMETRIC ARTICLE AND METHOD FOR ASSESSING NEUROLOGICAL AND PSYCHOLOGICAL BEHAVIORS THAT IMPACT DECISION MAKING

(71) Applicant: 3ethos, LLC, Stonington, CT (US)

(72) Inventor: Donald B. Trone, Stonington, CT (US)

(21) Appl. No.: 16/691,301

(22) Filed: Nov. 21, 2019

Related U.S. Application Data
(60) Provisional application No. 62/770,529, filed on Nov. 21, 2018.

(51) Int. Cl. G16H 50/00; G16H 10/20; G16H 15/00

(52) U.S. Cl. CPC G16H 50/00; G16H 15/00; G16H 10/20

(57) ABSTRACT

A system and method for assessing the neurological and psychological behaviors of an individual that impacts the decision-making process of the individual is provided, wherein the method includes performing an individual skill self-assessment, performing an individual effort self-assessment, determining outlier values for the individual skill self-assessment and/or the individual effort self-assessment, correcting the individual skill self-assessment and/or the individual effort self-assessment responsive to the outlier values and determining a final IMPACT score.

### RELATED APPLICATIONS

This application is related to and claims the benefit of U.S. Provisional Patent Application Ser. No. 62/770,529 filed Nov. 21, 2018, the contents of which are incorporated by reference herein in its entirety.

### FIELD OF THE INVENTION

The present invention relates generally to a method for assessing specific behaviors of an individual which impact the decision-making process of the individual and more particularly a method for assessing neurological and psychological behaviors that impact the decision-making process of an individual.

### BACKGROUND OF THE INVENTION

It is known that the vast majority of individuals and groups that face liability for the decisions that they make are not trained for the roles and responsibilities that they have assumed and typically do not have a defined, procedurally prudent decision-making process. As such, when failures or non-optimal results are blamed on the decisions of an individual or decision maker, the decision-making process of the decision maker is ultimately questioned and criticized. And when training is provided, it is often long and complex and the only part of the information is retained, if any information is retained at all. Furthermore, most of these decision makers are not aware of how their leadership and stewardship behaviors impact the quality of their decision-making process. This is undesirable because without fully understanding the characteristics of the behaviors that impact their decision-making process, the decision makers cannot begin to improve the quality, and thus impact, of their decisions.

### SUMMARY OF THE INVENTION

A method for assessing the neurological and psychological behaviors of an individual that impact the decision-making process of the individual is provide and includes performing an individual skill self-assessment, performing an individual effort self-assessment, determining outlier values for the individual skill self-assessment and/or the individual effort self-assessment, correcting the individual skill self-assessment and/or the individual effort self-assessment responsive to the outlier values and determining a final IMPACT score.

A system for assessing the neurological and psychological behaviors of an individual that impact the decision-making process of the individual is provided, wherein the system includes a data input device and a processing device communicated with the data input device. The processing device is configured to receive input data, process individual skill self-assessment data, process individual effort self-assessment data, determine outlier values for the individual skill self-assessment data and/or the individual effort self-assessment data, correct the individual skill self-assessment data and/or the individual effort self-assessment data responsive to the outlier values and determining a final IMPACT score.

### BRIEF DESCRIPTION OF THE DRAWINGS

The foregoing and other features and advantages of the present invention should be more fully understood from the accompanying detailed description of illustrative embodiments taken in conjunction with the following Figures in which like elements are numbered alike in the several Figures:

FIG. 1 illustrates an operational block diagram describing a method for assessing the neurological and psychological behaviors of an individual that impact the decision-making process of the individual, in accordance with one embodiment of the invention.

FIG. 2 is a screenshot illustrating one portion of an article for implementing the method of FIG. 1, in accordance with one embodiment of the invention.

FIG. 3 is a screenshot illustrating another portion of an article for implementing the method of FIG. 1, in accordance with one embodiment of the invention.

FIG. 4 is a screenshot illustrating the cover page of a report generated by implementing the method of FIG. 1, in accordance with one embodiment of the invention.

FIG. 5 is a screenshot illustrating another portion of a report generated by implementing the method of FIG. 1, in accordance with one embodiment of the invention.

FIG. 6A is a screenshot illustrating another portion of a report generated by implementing the method of FIG. 1, in accordance with one embodiment of the invention.

FIG. 6B is a screenshot illustrating one embodiment of a caution indicator when the IMPACT SCORE falls below a predefined level.

FIG. 7 is a screenshot illustrating another portion of a report generated by implementing the method of FIG. 1, in accordance with one embodiment of the invention.

FIG. 8 is a screenshot illustrating another portion of a report generated by implementing the method of FIG. 1, in accordance with one embodiment of the invention.

FIG. 9 is a screenshot illustrating another portion of a report generated by implementing the method of FIG. 1, in accordance with one embodiment of the invention.

FIG. 10 shows a portion of a questionnaire used to assess neurological and psychological behaviors of an individual, in accordance with one embodiment of the invention.

FIG. 11 shows a portion of the questionnaire of FIG. 10, in accordance with one embodiment of the invention.

FIG. 12 shows a portion of the questionnaire of FIG. 10, in accordance with one embodiment of the invention.

FIG. 13 shows a portion of the questionnaire of FIG. 10, in accordance with one embodiment of the invention.

FIG. 14 shows a portion of the questionnaire of FIG. 10, in accordance with one embodiment of the invention.

FIG. 15 shows a portion of the questionnaire of FIG. 10, in accordance with one embodiment of the invention.

FIG. 16 shows a portion of the questionnaire of FIG. 10, in accordance with one embodiment of the invention.

FIG. 17 shows a portion of a questionnaire used to assess neurological and psychological behaviors of a group, in accordance with one embodiment of the invention.

FIG. 18 shows a portion of the questionnaire of FIG. 17, in accordance with one embodiment of the invention.

FIG. 19 shows a portion of the questionnaire of FIG. 17, in accordance with one embodiment of the invention.

FIG. 20 shows a portion of the questionnaire of FIG. 17, in accordance with one embodiment of the invention.

FIG. 21 shows a portion of the questionnaire of FIG. 17, in accordance with one embodiment of the invention.

FIG. 22 shows a portion of the questionnaire of FIG. 17, in accordance with one embodiment of the invention.

FIG. 23 shows a portion of the questionnaire of FIG. 17, in accordance with one embodiment of the invention.

FIG. 24 illustrates an algorithm for the calculation of the IMPACT score and outliers, in accordance with one embodiment of the invention.

FIG. 25 illustrates a portion of the algorithm of FIG. 24, in accordance with one embodiment of the invention.

FIG. 26 illustrates a summary of the assessment, in accordance with one embodiment of the invention.

### DETAILED DESCRIPTION

It is well known that there are certain people whose brains, through development, are better wired to lead. They found that these types of people have a propensity to rise to leadership roles and share similar neurological and psychological factors. It should be appreciated that the present invention accelerates the professional development of individuals and/or groups (teams, staff, boards or committees) that have a legal, financial, professional and/or moral liability for their decision-making process, for example executives, politicians, fiduciaries, directors, officers, investment committee members, consultants, advisors and senior staff.

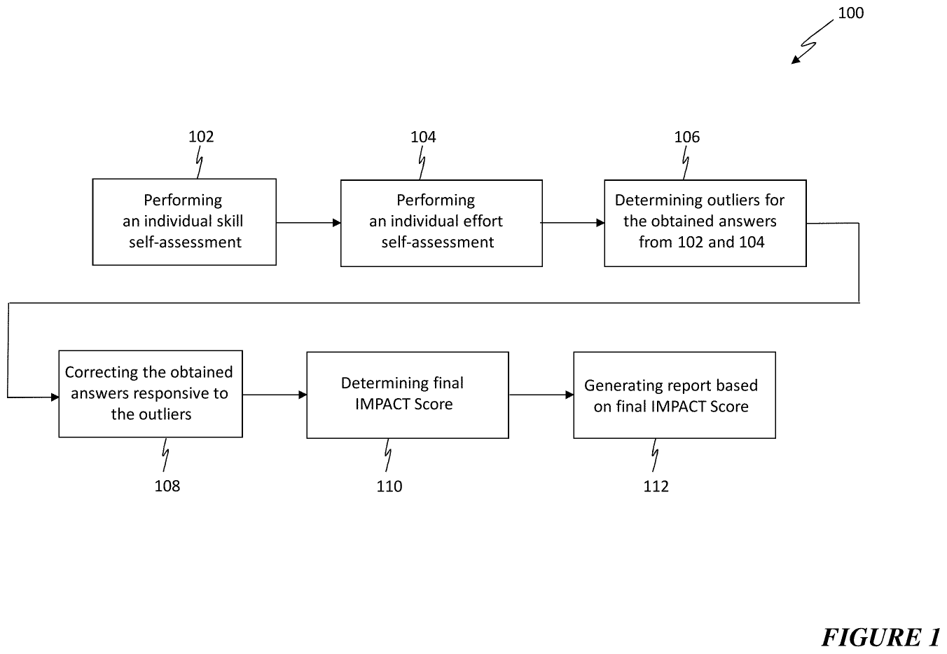

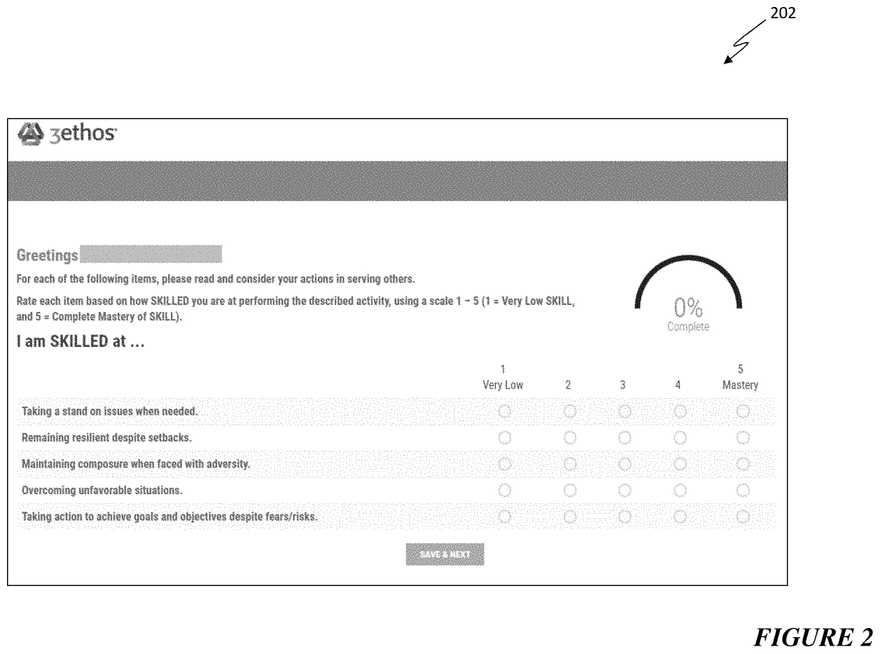

Referring to the FIGs., a method and system for assessing the neurological and psychological behaviors of an individual that impact the decision-making process of the individual is provided. Referring to FIG. 1, an operational block diagram illustrating a method 100 for assessing the neurological and psychological behaviors of an individual that impact the decision-making process of the individual is shown, in accordance with one embodiment of the invention. The method 200 includes performing a predefined Individual Skill Self-Assessment (ISSA) 202 which is configured to assess the skill of the individual, as shown in operational block 102, wherein the ISSA 202 is assessed against a predefined level scale, for example 1-5, where 5 is the maximum. One example of some questions used by the ISSA 202 is shown in FIG. 2. The ISSA 202 may include multiple questions that are answered by the individual taking the assessment and which are tailored to assess leadership behaviors, for example, the courageousness of the individual, the ability/willingness to collaborate with others, fullness of character, ability/willingness to show compassion and/or competency of the individual. The ISSA 202 may also be tailored to assess stewardship behaviors, for example, the individual's ability to Align, Adapt, be Attentive, Authenticity and/or Accountability. The ISSA 202 may also be further tailored to assess governance behaviors, for example, the individual's ability to Analyze, Strategize, Formalize, Implement and Monitor. One embodiment of questions used by the ISSA 202 are shown and may include about seventy-five (75) and/or less or more questions that are configured to assess specific characteristics related to skill.

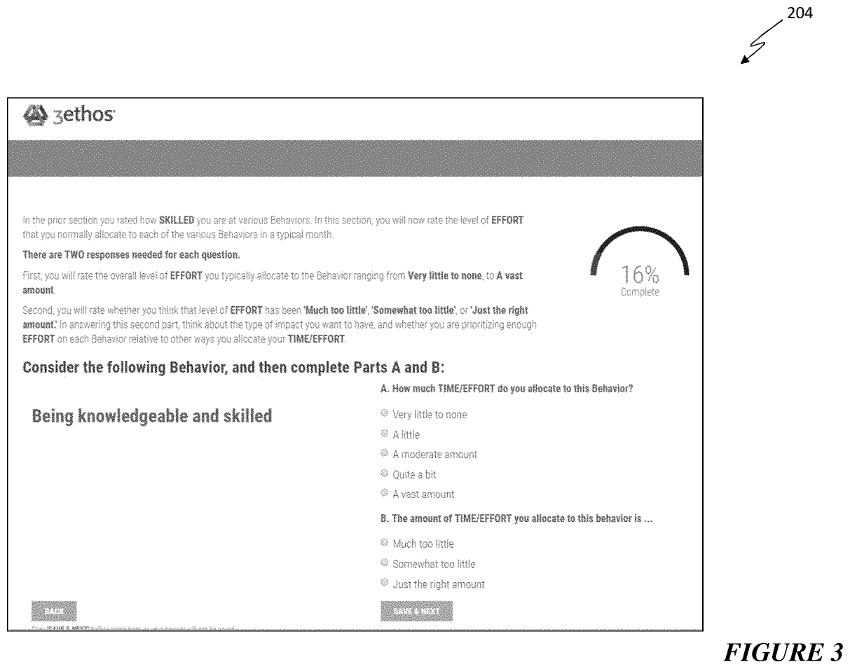

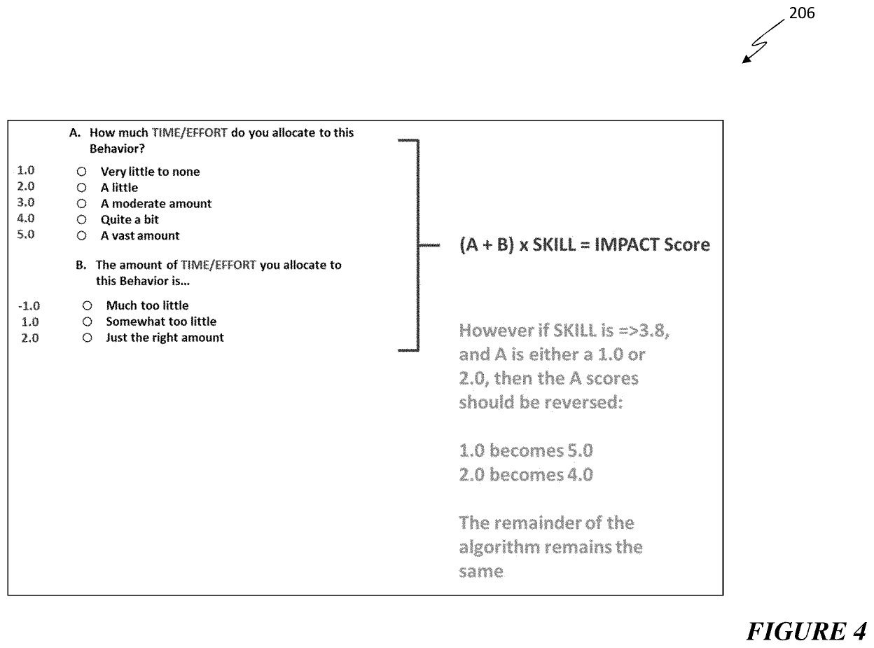

Referring to FIG. 3, the method 200 also includes performing a predefined Individual Effort Self-Assessment (IESA) 204 which is configured to assess the effort spent by the individual on a particular skill, as shown in operational block 104, wherein the IESA 204 may be assessed using at least one of the two metrics. One of the metrics reflects what the individual thinks about the amount of time the individual spent on a particular skill, where the metric is rated from “Much too little” to “Just the right amount,” and the other metric is whether the individual believes that the amount of time spent is appropriate where the metric is rated from “Very little” to “A vast amount.” Referring to FIG. 4, the responses and ratings are then processed to determine a preliminary IMPACT SCORE for the five (5) leadership behaviors, five (5) stewardship behaviors and five (5) governance behaviors. It should be appreciated that, in one embodiment, the preliminary IMPACT SCORE may be calculated by multiplying the Skill rating times the Effort rating, where the Effort rating is converted to a numerical value using the algorithm as disclosed herein. One embodiment of the IESA questions are shown and include about fifteen (15) questions that are configured to assess specific characteristics related to effort.

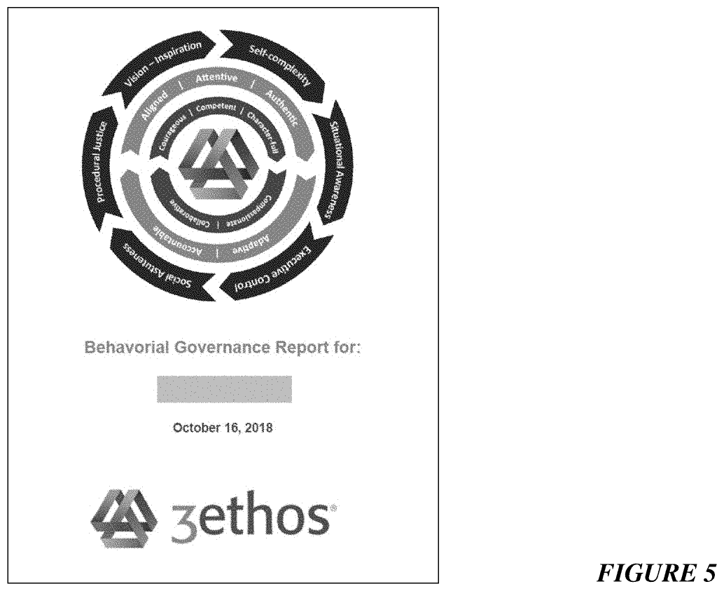

The method 200 further includes determining outliers for the answers provided, as shown in operational block 106, and correcting the outliers, as shown in operational block 108, wherein correcting the outliers includes processing the outliers via a predetermined algorithm as provided herein. A final IMPACT SCORE for the five (5) leadership behaviors, five (5) stewardship behaviors and five (5) governance behaviors is then determined, as shown in operational block 110, where the final IMPACT SCORE is responsive to and reflects a two-dimensional view of both Skill and Effort. As described in more detail hereinafter, the method 200 also includes generating a report which identifies to the individual which areas need improvement and how they can improve, as shown in operational block 112. Referring to FIGS. 5-8, a portion of one embodiment of a report is shown.

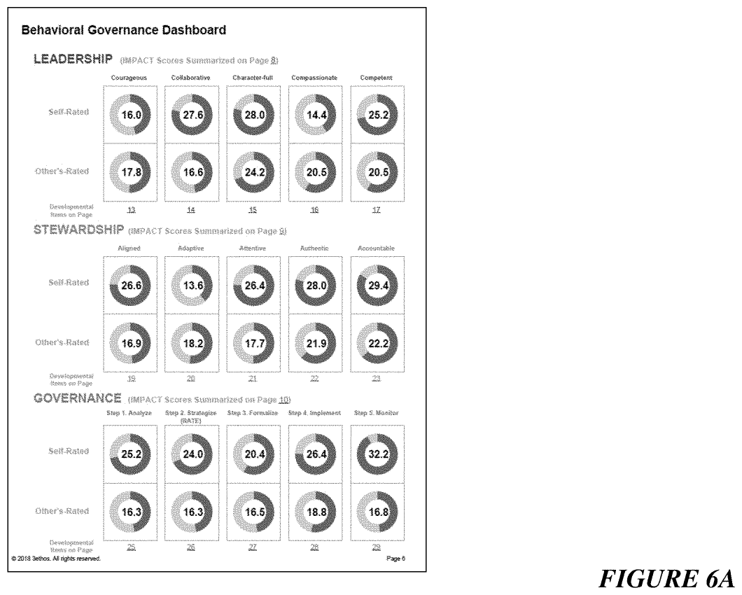

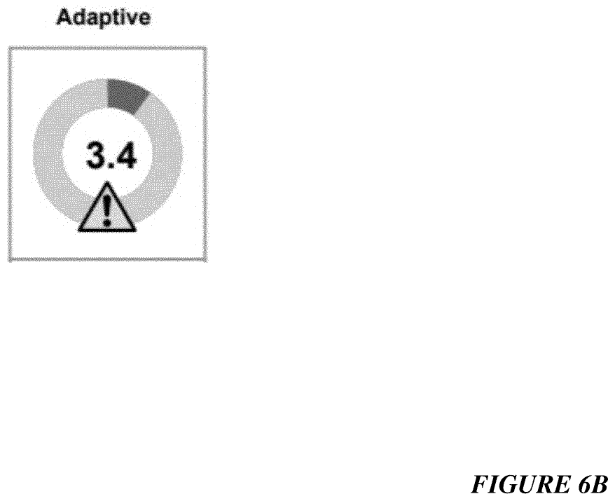

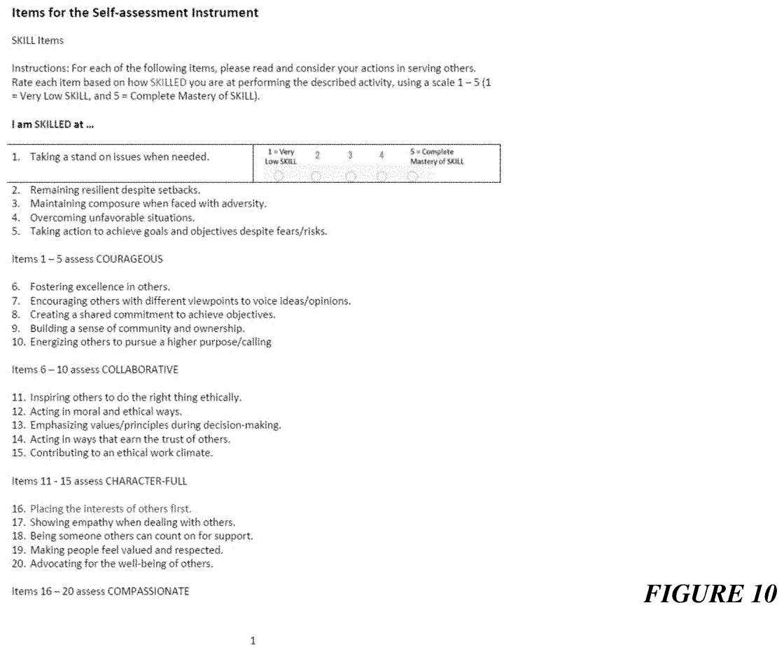

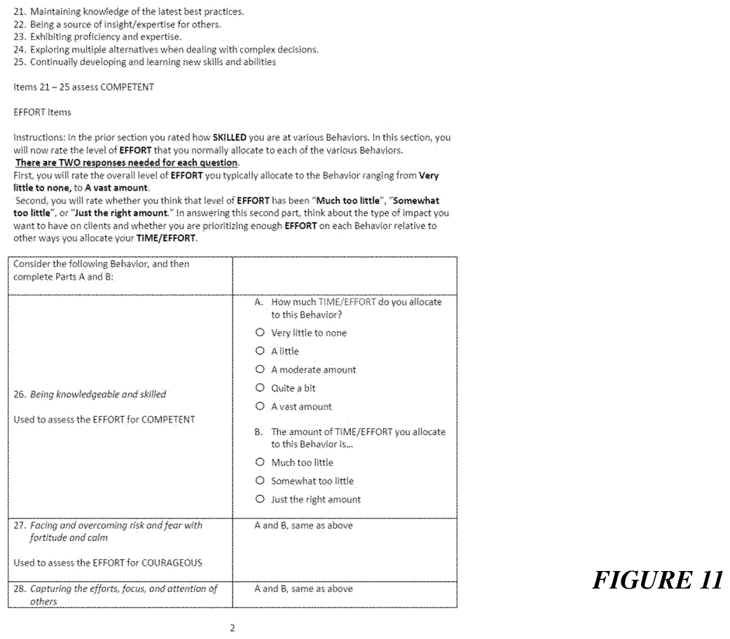

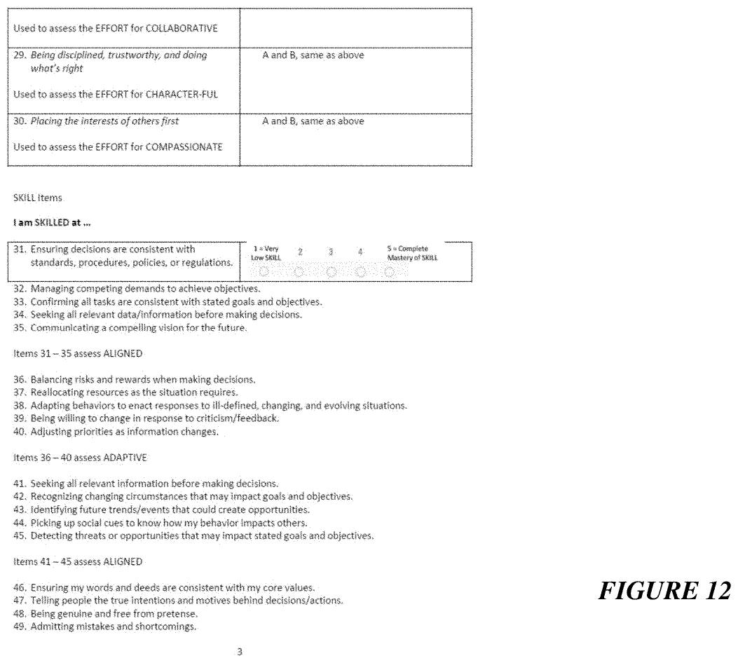

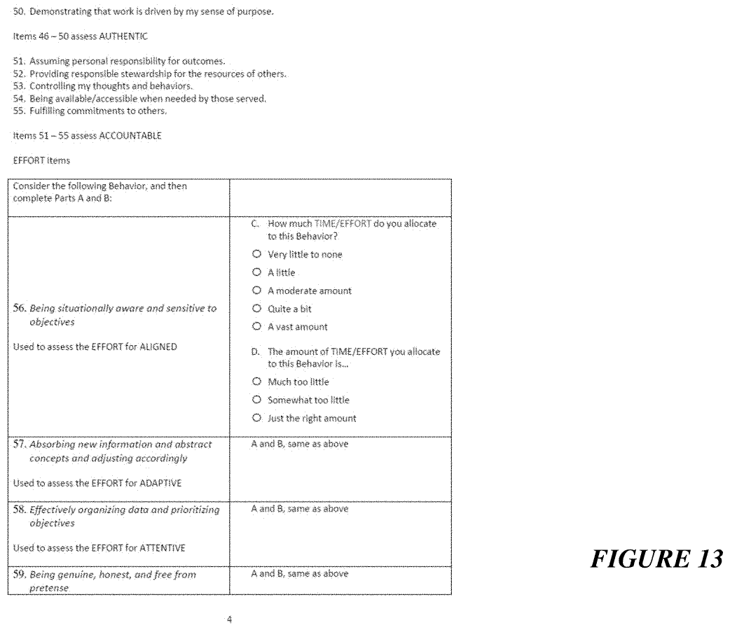

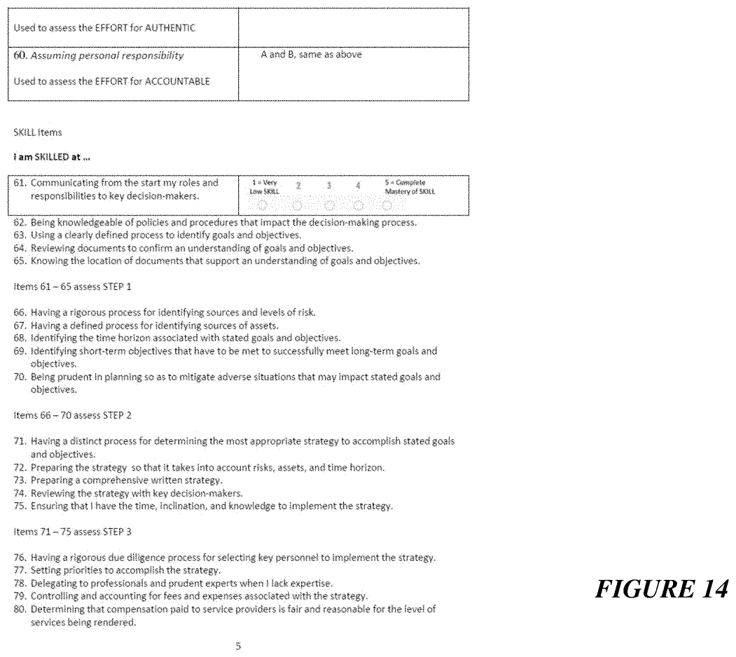

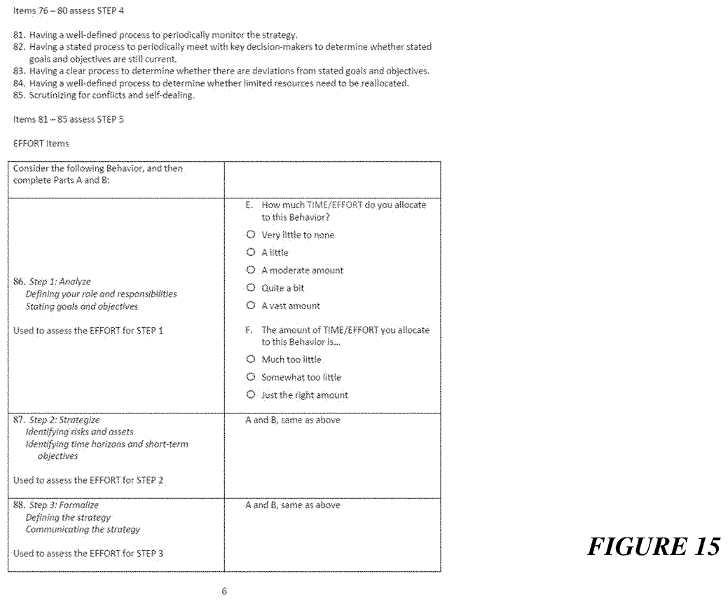

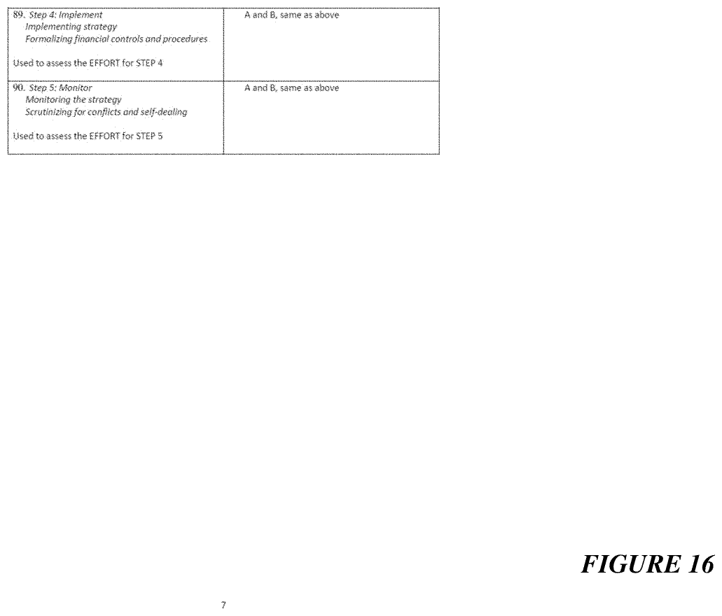

In accordance with one embodiment of the invention, the IMPACT SCORE may be calculated as described herein. Referring to FIG. 6A, it should be appreciated that in accordance with one embodiment, there are fifteen (15) IMPACT scores: 5 for Leadership, 5 for Stewardship and 5 for Governance. Referring to FIGS. 10-16, one embodiment of a questionnaire for conducting an ISSA 202 and an IESA 204 is shown and includes a plurality of questions used to assess an individual. It should be appreciated that the questionnaire includes ninety (90) assessment questions, wherein Questions 1-25 correspond to Leadership behavior, Questions 31-55 correspond to Stewardship behavior and Questions 61-85 correspond to Governance Behavior or decision-making skills. With regards to Questions 1-25, questions 1-5 correspond to Courageous, questions 6-10 correspond to Collaborative, questions 11-15 correspond to Character-Full, questions 16-20 correspond to Compassionate and questions 21-25 correspond to Competency. With regards to Questions 31-55, questions 31-35 correspond to Aligned, questions 36-40 correspond to Adaptive, questions 41-45 correspond to Attentive, questions 46-50 correspond to Authentic and questions 51-55 correspond to Accountable. With regards to Questions 61-85, questions 61-65 correspond to Analyze, questions 66-70 correspond to Strategize, questions 71-75 correspond to Formalize, questions 76-80 correspond to Implement and questions 81-85 correspond to Monitor.

It should be appreciated that each of the questions within the groups of questions are then answered based on a scale of 1-5, where 1 is rated Very Low Skill and 5 is rated Complete Mastery of Skill (although any scaling convention may be used). Thus, each of the groups of 5 (five) questions then have 5 (five) individual scores (one for each question). The individual scores for each of the groups of 5 (five) questions are then averaged to produce fifteen (15) group scores; one (1) group score for each of the groups of 5 (five) questions (See FIG. 6A). Each of the group scores are then rated for the amount of time/effort allocated to the associated behavior and for whether the amount time/effort allocated to the associated behavior was “much too little,” “somewhat too little,” or “just the right amount,” where “much too little” “somewhat too little,” or “just the right amount.”

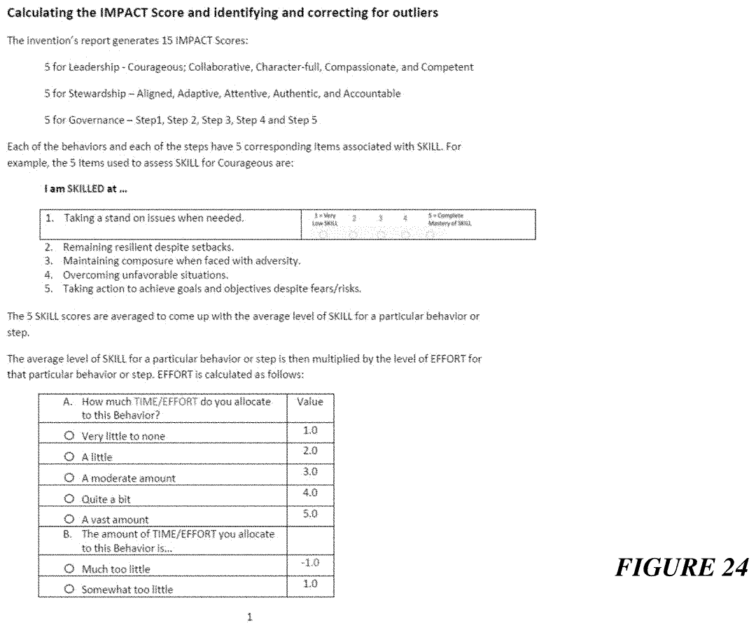

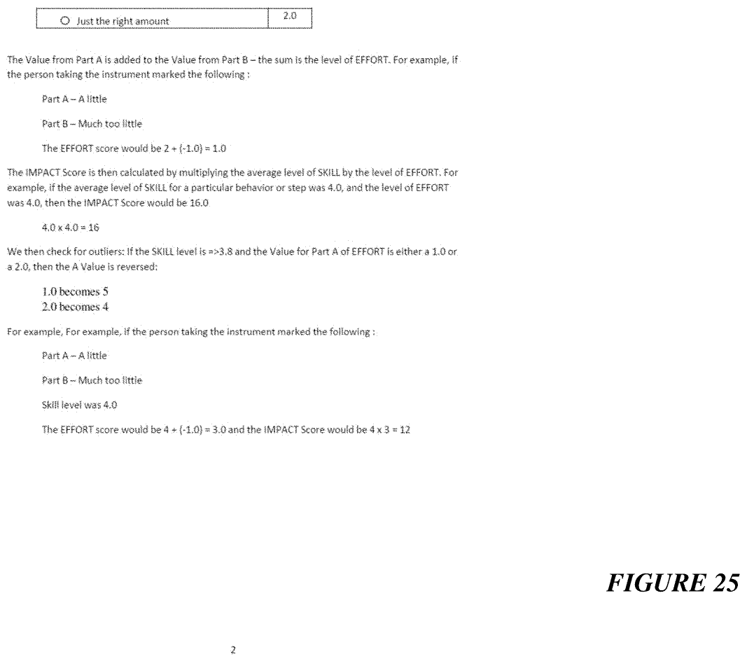

Each of the group of questions are then processed for a group A result and a Group B result. The group A result may be obtained by rating each of the group of questions based on the amount of time/effort spent on a particular Skill (represented by the group of questions). In one embodiment, the choices may be “Very little to none,” “A little,” “A moderate amount,” “Quite a bit” and “A vast amount,” where “Very little to none” is assigned a numerical value of 1.0, “A little” is assigned a numerical value of 2.0, “A moderate amount” is assigned a numerical value of 3.0, “Quite a bit” is assigned a numerical value of 4.0 and “A vast amount” is assigned a numerical value of 5.0. The group B result may be obtained by rating each of the group of questions based on whether the amount of time/effort spent on a particular Skill (represented by the group of questions) was felt to be “Much too little,” “Somewhat too little” or “Just the right amount,” where “Much too little” is assigned a numerical value of −1.0, “Somewhat too little” is assigned a numerical value of 1.0 and “Just the right amount” is assigned a numerical value of 2.0. For each of the group of questions, the group A result is added to the group B result to obtain a numerical value that represents the Level of Effort (LoE) for a particular skill. Referring to FIGS. 24-25, the preliminary IMPACT Score for each group is then calculated by multiplying the average level of Skill for each of the fifteen (15) groups by the numerical value assigned to the Level of Effort (LoE) rated for that particular behavior or step.

At this point, the final IMPACT Score for each of the groups may be determined by identifying outliers and correcting for these outliers. The outlier for each group may be determined by identifying if the Skill level for the group is greater than or equal to (=>) 3.8 and if the group A result is either a 1.0 or a 2.0. If these conditions are true, then the group A result is reversed in order, .i.e. 1.0 becomes 5.0 and 2.0 becomes 4.0. This is illustrated by the following example: If the Skill level for a particular group is rated at 4.0, and the group A result is rated at 2.0 and the group B result is rated at −1, then the Group A result is reversed in order such that the group A result is now rated at 4.0. Therefore, the final IMPACT Score is:

=(Group A result (adjusted for outlier)+Group B result)*Skill Level; or Final IMPACT Score=(4.0+(−1))*4.0=12

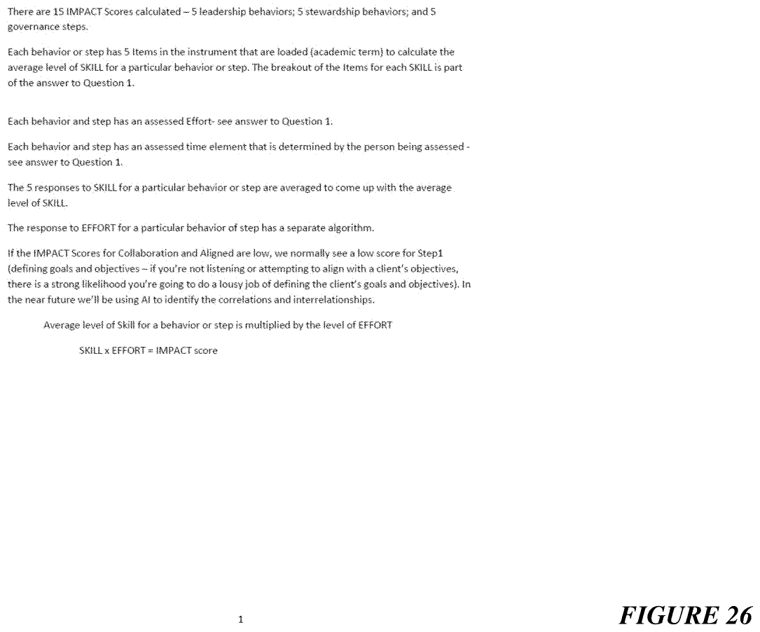

As briefly discussed above and in accordance with one embodiment of the invention, once the results have been generated, the method 200 includes generating a report which identifies to the individual which areas need improvement and how they can improve. Referring to FIG. 6A and FIG. 26, the report may include a dashboard that shows the 15 IMPACT SCORES (5 leadership, 5 stewardship and 5 governance) as well as the 15 IMPACT SCORES of “Others” who have taken the same instrument. If desired, the “Others” group of IMPACT SCORES can be set by the person administering the instruments and reports or may be automatically generated as desired either randomly or via predetermined criteria. In one embodiment, the IMPACT SCORES are depicted in the form of a radial button, where the radial button is filled in to depict the percentage of the ratio between the IMPACT SCORE and 35. It should be appreciated that although in one embodiment the report does not provide any indications that may make the individual feel defensive, such as color coding—red, yellow or green; a 100 point scale; or Pass/Fail, in another embodiment these indications may be used. It should be noted that, as shown in FIG. 6B, the report may (or may not) use a caution sign any time an IMPACT SCORE=<8.0 (or other predefined level).

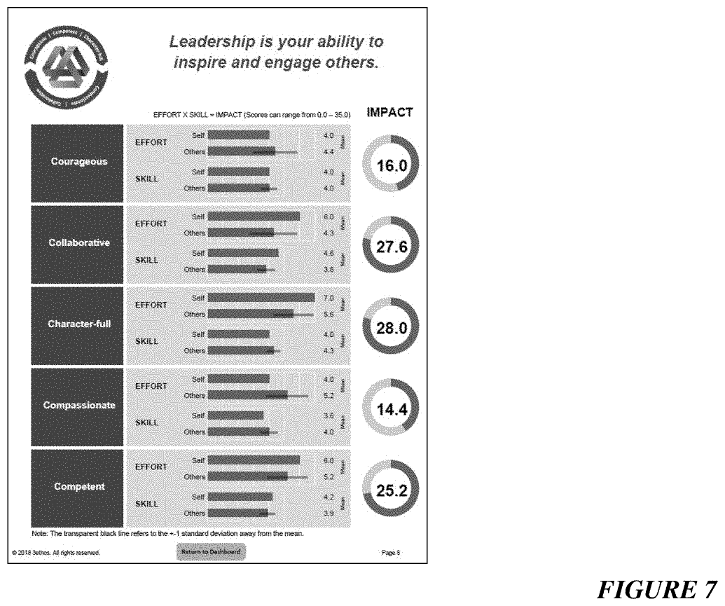

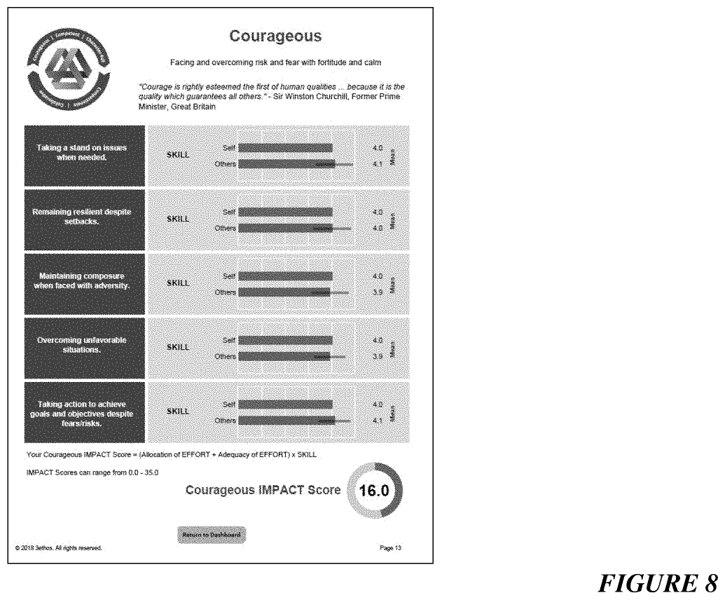

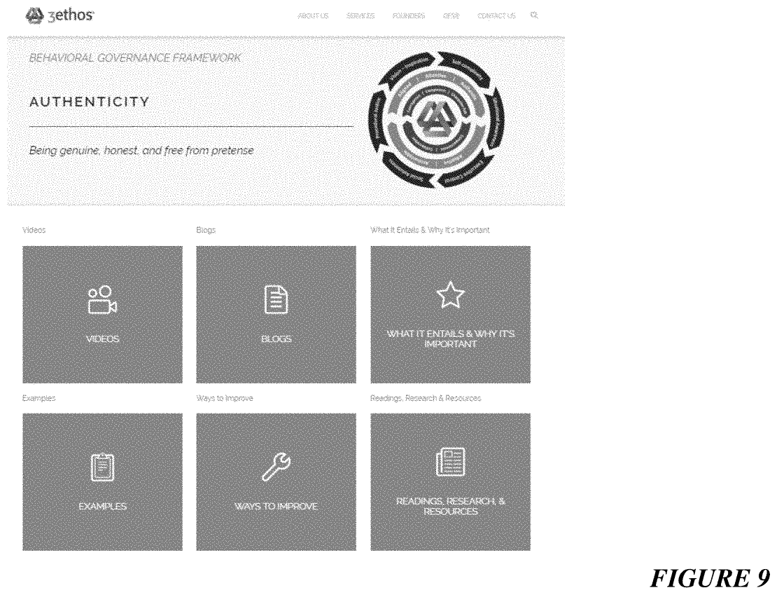

It should be appreciated that the dashboard may also include hyperlinks so that the person or group being assessed can (1) learn more about a particular IMPACT Score; and/or, (2) the items that were loaded (associated) with a particular SKILL. One such embodiment is shown in FIG. 7 and FIG. 8. A person wanting to further develop any behavior or governance dimension can click on any of the radial buttons and be taken to a separate landing page (website) that contains content specific to the behavior or governance step, as shown in FIG. 9. It should be appreciated that the landing pages may provide the person or group being assessed with an AL (Augmented Learning) experience. In one embodiment the person or group can select the features they want to include in their developmental plan; such as: videos, blogs, checklists, research, and/or books. In the case of the behaviors, there may be a detailed explanation from neuroscientists on why the particular behavior is important. In the case of the governance steps there may be a detailed explanation of the best practices associated with that step. For investment fiduciaries, there may also be legal substantiation.

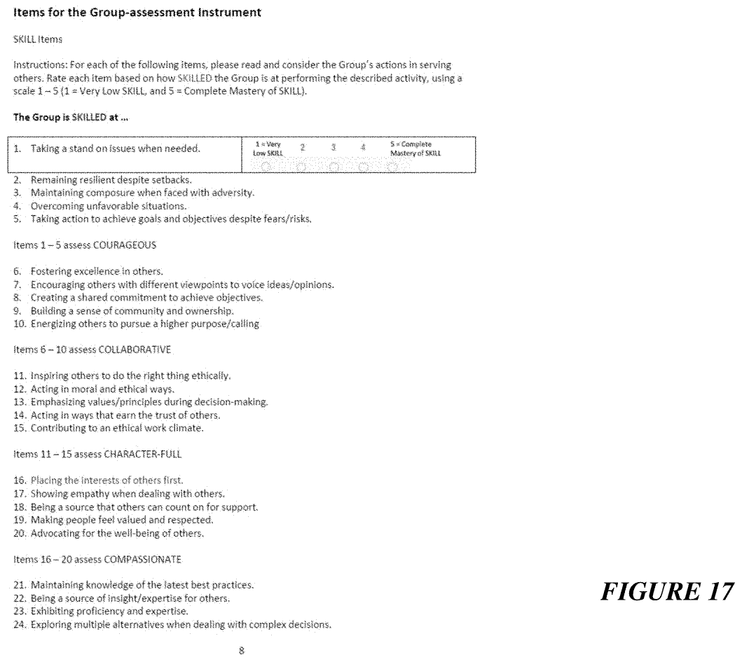

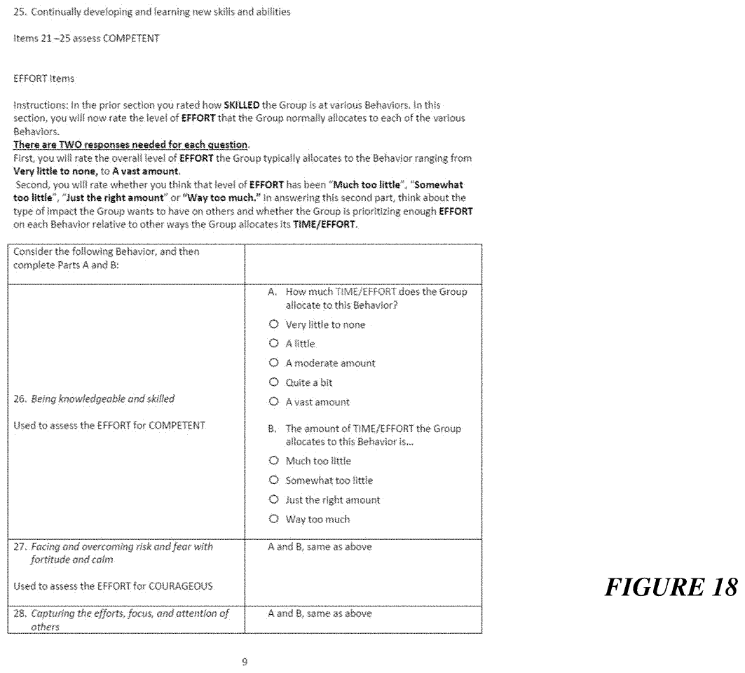

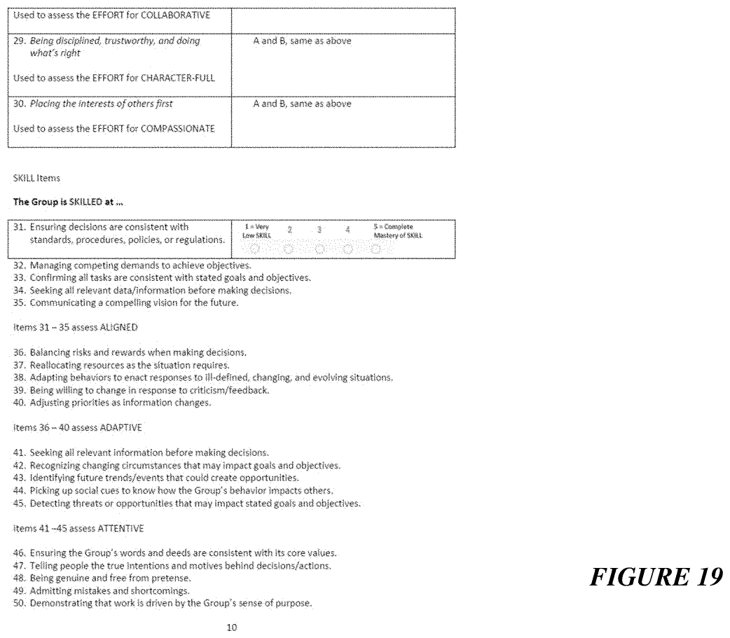

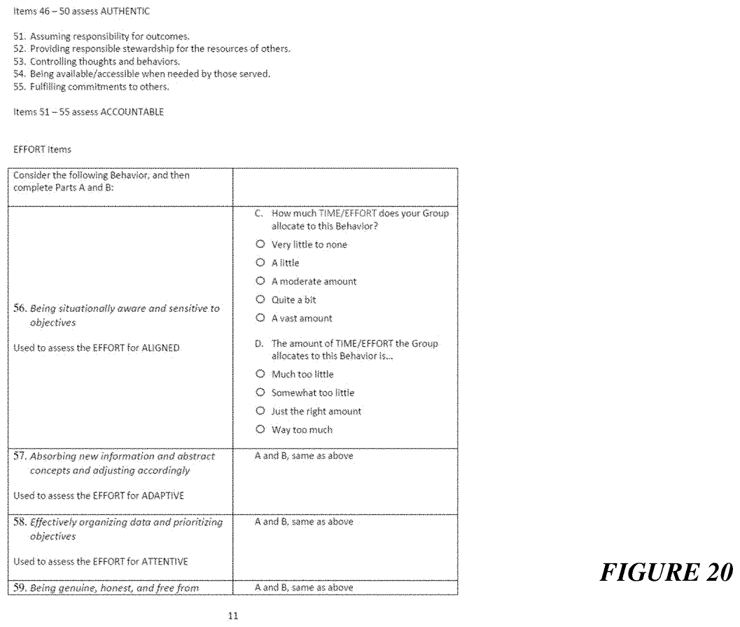

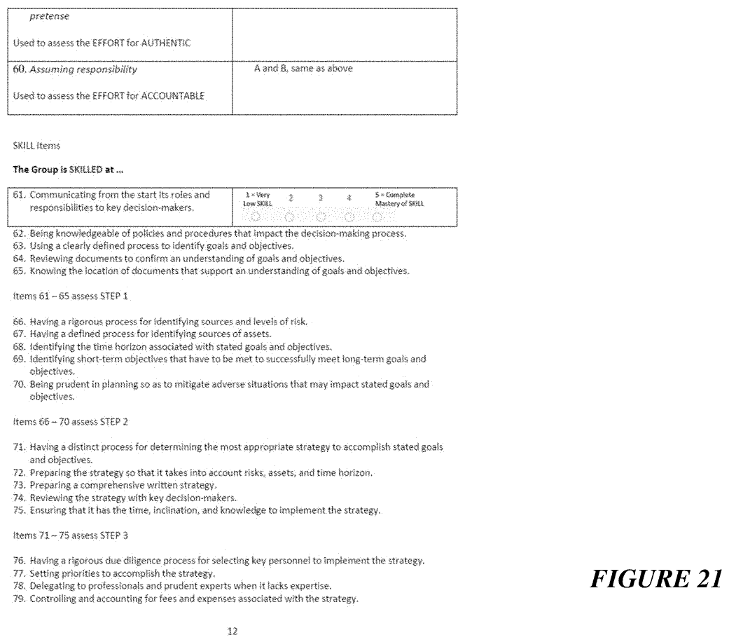

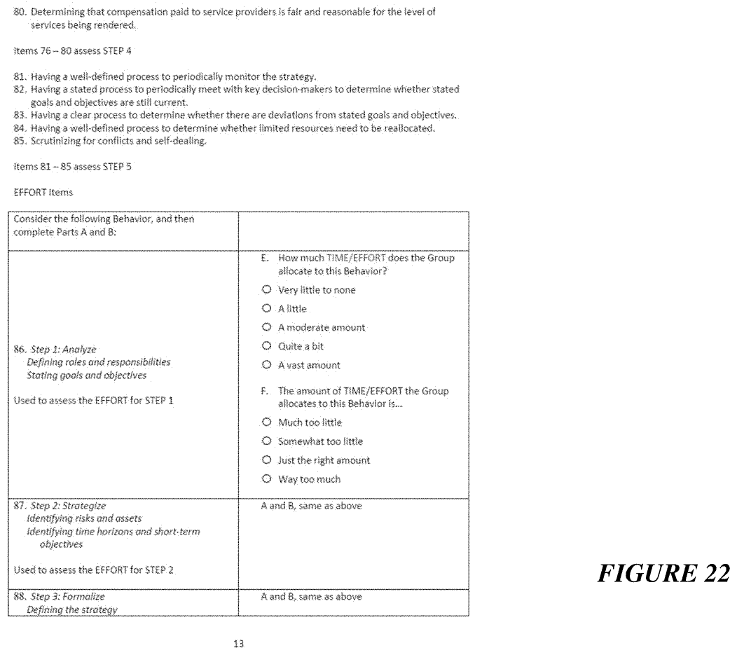

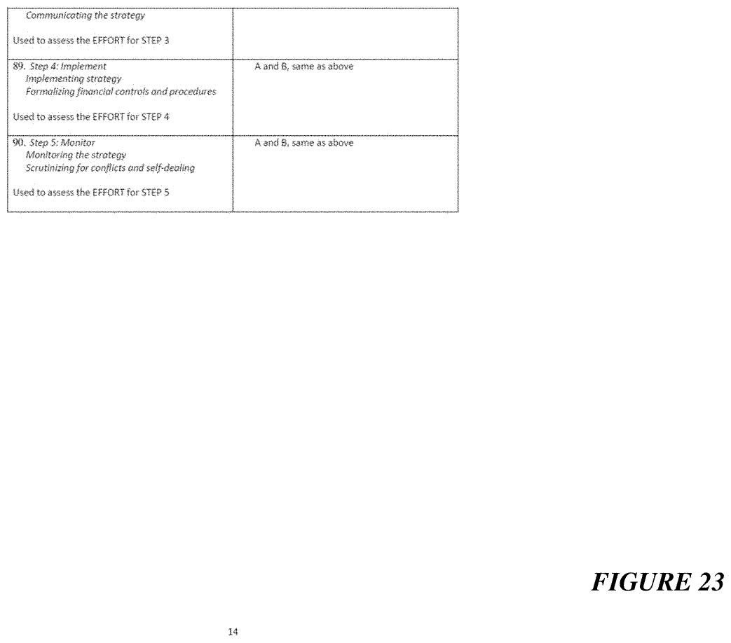

In addition to individual assessment, referring to FIGS. 17-23, it should be appreciated that the method 100 of the present invention can be applied to group assessment, as well as a 360-degree assessment. In accordance with one embodiment, some example questions that may be used for the group assessment are also shown.

As described above, the methods and embodiments described hereinabove and in the several figures may be embodied in the form of computer-implemented processes and apparatuses for practicing those processes. The methods and embodiments described hereinabove and in the several figures may also be embodied in the form of computer program code containing instructions embodied in tangible media, such as floppy diskettes, CD-ROMs, hard drives, or any other computer-readable storage medium, wherein, when the computer program code is loaded into and executed by a computer, the computer becomes an apparatus for practicing the invention. Existing systems having reprogrammable storage (e.g., flash memory) may be updated to implement the invention. The methods and embodiments described hereinabove and in the several figures may also be embodied in the form of computer program code, for example, whether stored in a storage medium, loaded into and/or executed by a computer, or transmitted over some transmission medium, such as over electrical wiring or cabling, through fiber optics, or via electromagnetic radiation, wherein, when the computer program code is loaded into and executed by a computer, the computer becomes an apparatus for practicing the invention. When implemented on a general-purpose microprocessor, the computer program code segments may configure the microprocessor to create specific logic circuits. It should be further appreciated that the methods and embodiments described hereinabove may also be practiced, in whole or in part, via any device suitable to the desired end purpose, such as a computer, iPod, MP3 Player, a PDA, a Pocket PC and/or a Cell phone with connection capability.

While the invention has been described with reference to an exemplary embodiment, it should be understood by those skilled in the art that various changes may be made and equivalents may be substituted for elements thereof without departing from the scope of the invention. Moreover, the embodiments or parts of the embodiments may be combined in whole or in part without departing from the scope of the invention. In addition, many modifications may be made to adapt a particular situation or material to the teachings of the invention without departing from the scope thereof. Therefore, it is intended that the invention not be limited to the particular embodiment disclosed as the best mode contemplated for carrying out this invention, but that the invention will include all embodiments falling within the scope of the appended claims. Moreover, unless specifically stated any use of the terms first, second, etc. do not denote any order or importance, but rather the terms first, second, etc. are used to distinguish one element from another.

I claim:

1. A method for assessing the neurological and psychological behaviors of an individual that impact the decision-making process of the individual, the method comprising:
performing an individual skill self-assessment;
performing an individual effort self-assessment;
determining outlier values for the individual skill self-assessment and/or the individual effort self-assessment;
correcting the individual skill self-assessment and/or the individual effort self-assessment responsive to the outlier values; and
determining a final IMPACT score.

2. The method of claim 1, further including generating a report based on the final IMPACT score.

3. A system for assessing the neurological and psychological behaviors of an individual that impact the decision-making process of the individual, the system comprising:
a data input device; and
a processing device communicated with the data input device, wherein the processing device is configured to receive input data, process individual skill self-assessment data;
process individual effort self-assessment data;
determine outlier values for the individual skill self-assessment data and/or the individual effort self-assessment data;
correct the individual skill self-assessment data and/or the individual effort self-assessment data responsive to the outlier values; and
determining a final IMPACT score.

4. The system of claim 3, wherein the processing device is configured to generate report based on the final IMPACT score.
